(19) United States
(12) Patent Application Publication
(10) Pub. No.: US 2004/0029195 A1
(43) Pub. Date: Feb. 12, 2004

(54) Compositions and methods for cancer screening

(76) Inventor: Ramin Shiekhattar, Philadelphia, PA (US)

Correspondence Address: LICATLA & TYRRELL P.C., 66 E. MAIN STREET, MARLTON, NJ, 08053, US

(21) Appl. No.: 10/333,543

(22) PCT Filed: Jul. 19, 2001
(86) PCT No.: PCT/US01/22684

(51) Int. Cl. G01N 33/574; A61K 39/395; C07K 16/30

(52) U.S. Cl. 435/7.23; 424/155.1; 530/388.8

(57) ABSTRACT

Methods for screening for cancer are provided which are based on the direct interaction of BRCA1 with the mammalian SWI/SNF complex through association with its BRG1 subunit. Also provided are compositions for modulating cellular proliferation by modulating chromatin remodeling activity in mammalian cells which targets a BRG1 subunit of the SWI/SNF complex.

### BACKGROUND OF THE INVENTION

BRCA1 is a tumor suppressor gene that is linked to familial breast and ovarian cancers. The BRCA1 gene encodes a protein of 1,863 amino acids which has a primary sequence containing two motifs characteristic of transcription factors, a RING-finger motif and an acidic carboxyl terminus. Fusions of the carboxyl terminus to the DNA binding domain of the GAL4 protein endows the chimeric protein with transcriptional stimulatory activity (Chapman, M. S. and I. M. Verma. 1996. Nature 382:678-679; Monteiro, A. N. et al. 1996. Proc. Natl. Acad. Sci. 26:13595-13599). BRCA1 has been shown not only to function as a coactivator for p53-mediated transcription but also its overexpression has been shown to result in enhancement of p53-independent gene expression (Ouchi, T. et al. 1998. Proc. Natl. Acad. Sci. USA 95:2302-2306; Somasundaram, K. et al. 1997. Nature 389:187-190). BRCA1 has also been reported to be a component of an RNA polymerase II complex (Scully, R. et al. 1997. Proc. Natl. Acad. Sci. USA 94:5605-5610). These data suggest that BRCAL has function as a transcriptional regulator.

Other data have implicated BRCA1 in DNA repair mechanisms. BRCA1 has been reported to interact with RAD51, BRCA2 and the RAD50 protein complex (Chen, J. et al. 1998. Mol. Cell 2:317-328; Zhong, Q. et al. 1999. Science 285:747-750; Gowen, L. C. et al. 1998. Science 281:1009-1012; Scully, R. et al. 1997. Cell 88:265-275; Scully, R. et al. 1999. Mol. Cell 4:1093-1099). Moreover, BRCA1 mutant cells display sensitivity to DNA damaging agents and the BRCAL protein was reported to control homology-directed DNA repair (Moynahan, M. E. et al. 1999. Mol. Cell 4:511-518). Truncation of BRCA1 exon 11 has also been shown to result in defective G2-M cell cycle check point and increased number of centrosomes (Xu, X. et al. 1999. Mol. Cell 3:389-395).

Several transcriptional regulatory complexes have been identified in mammalian cells. Among these are the RNA polymerase II (polII) holoenzyme complex and the SWI/SNF complex. Altered activity of such complexes have been suggested to be linked to development of cancer (Wolffe, A. P. 1999. Science Med. 6:28-37). The mammalian PolII holoenzyme has been shown to be associated with cancer-associated genes such as the tumor suppressor gene BRCA1 (Neish, A. S. et al. 1998. Nucleic Acids res. 26:847-853).

It has now been found that BRCA1 is directly associated in mammalian cells with the human SWI/SNF complex through interaction with the complex's BRG1 subunit.

### SUMMARY OF THE INVENTION

An object of the present invention is a composition for modulating cellular proliferation by modulating chromatin remodeling activity in mammalian cells which targets a BRG1 subunit of the SWI/SNF complex.

Another object of the present invention is a method for modulating cellular proliferation by modulating chromatin remodeling activity in mammalian cells which comprises interacting BRCA1 with a composition which targets a BRG1 subunit of the SWI/SNF complex.

Another object of the present invention is a method of screening for cancer which comprises identifying the presence of mutations in the BRG1 subunit and other subunits of the SWI/SNF complex in a biological sample.

Yet another object of the present invention is a method of modulating transcriptional activity in a mammalian cell which comprises interacting BRCA1 with the BRG1 subunit of mammalian SWI/SNF protein complex and thereby modulating the chromatin-remodeling activity in said cell so that transcription, and thereby cellular proliferation, is modulated.

Another object of the present invention is a composition for modulating estrogen signaling in mammalian cells which targets a BRG1 subunit of the SWI/SNF complex.

### DETAILED DESCRIPTION OF THE INVENTION

The present invention is a method of modulating transcriptional activity in a mammalian cell which involves interaction of BRCA1 with the BRG1 subunit of a mammalian SWI/SNF complex where this interaction leads to changes in or modulation of the chromatin-remodeling activity of the cell and thus transcription is modulated and ultimately cellular proliferation is modulated. In the context of the present invention, “modulating” is meant to refer to either an increase or a decrease in activity. Since mutations in other subunits of the mammalian SWI/SNF complex have been shown to result in certain forms of cancer (e.g., Versteege, I. et al. 1998. Nature 394:203-206), the present invention is also a method for screening for cancer in animals, including humans, which comprises identifying the presence of a mutation in the BRG1 subunit of the SWI/SNF complex. In the context of the present invention screening for cancer is meant to include both diagnosis of existing cancer and identification of individuals at risk for developing cancer.

The present invention is also a composition for modulating cellular proliferation by increasing chromatin remodeling activity in mammalian cells that acts by targeting the BRG1 subunit of the SWI/SNF complex. Since chromatin remodeling in cells depends on the recruitment of enzymes that control the coiling of DNA, and chromatin remodeling itself can lead to activation or inactivation of gene expression in a cell (Wolffe, A. P. 1999. Science Med. 6:28-37), the present invention is also a composition that can affect activation of genes that cause cancer through cellular proliferation. Therefore, one of skill would understand that the present invention is a method for modulating cellular proliferation.

It has now been found that BRCA1 is a component of a human SWI/SNF-related complex. Further, it has been found for the first time that BRCA1 interacts specifically with the BRG1 subunit of the mammalian SWI/SNF complex, an interaction that results in a modulation of chromatin structure, a modulation of transcription, and ultimately a modulation of cellular proliferation. Therefore, the present invention is a method for modulating transcriptional activity and cellular proliferation in a cell.

Experiments were performed first to purify a BRCA1 transcriptional control complex. To determine the polypeptide composition of endogenous BRCA1-containing complexes, HeLa cell nuclear extract was chromatographed sequentially to enrich for BRCA1-containing complexes. Analysis of these fractions by gel filtration chromatography revealed that BRCA1 is a component of a 2 MDA complex. To purify this complex, DEAE-Sephacel fractions were subjected to affinity-purification using polyclonal anti-BRCA1 antibodies. Polypeptides specifically eluted from the BRCA1 affinity matrix were then sequences by ion trap tandem mass spectrometry. Twenty-six tryptic peptide sequences confirmed the identity of the 210 kDa polypeptide as BRCA1 indicating that the purification was specific.

Additionally, five peptide sequences identified a 180 kDa polypeptide as human BRG1 protein. A 60 kDa polypeptide contained three peptide sequences corresponding to BAF60b. BRG1 and BAF60 have been reported to be components of a multi-protein human chromatin remodeling complex known as SWI/SNF (Wang, W. et al. 1996. Embo J. 15:5370-5382; Wang, W. et al. 1996. Genes Dev. 10:2117-2130). Western blot analysis of the BRCA1 affinity eluate revealed the presence of three other SWI/SNF components, SNF5, BAF155 and BAF170. The SWI/SNF complex is known to display chromatin remodeling activity as measured by changes in the DNASE I digestion pattern of mononucleosomes in an ATP-dependent manner (Cote, J. et al. 1994. Science 265:53-60; Wang, W. et al. 1996. EMBO J. 15:5370-5382; Bochar, D. A. et al. 2000. Proc. Natl. Acad. Sci. USA 97:1038-1043). Thus, to further establish the association between BRCA1 and SWI/SNF complex, the BRCA1-affinity eluate was tested in a mononucleosome digestion assay. Results showed that affinity-purified BRCA1 complex displayed chromatin remodeling activity by altering the ATP-dependent DNase I cleavage of mononucleosome to a pattern more closely resembling that of naked DNA. These data demonstrated that there is a physical association between the BRCA1 protein and the SWI/SNF remodeling complex, an association that leads to physiological activity.

Since BRCA1 has been reported to be associated with a number of other multi-protein complexes, the association of ERCA1 with SWI/SNF was examined further to determine if it is a dominant form in humans. “Dominant” in the context of this invention denotes that its occurrence is common rather than rare. To determine the number of BRCA1-containing complexes that might be present, BRCA1 complexes were purified to near homogeneity by fractionating an HeLa nuclear extract. Western blot analysis of column fractions throughout the entire purification revealed the precise co-elution of BRCA1 with components of the SWI/SNF complex. Only a single peak of BRCA1 immunoreactivity was detected on the column, a peak that co-eluted with components of the SWI/SNF complex. These data indicated that BRCA1-SWI/SNF represents the predominant BRCA1-containing complex in HeLa nuclear extract. Similar to the affinity-purified BRCA1-SWI/SNF complex, the conventionally purified complex displayed mononucleosome disruption activity. In addition, the BRCA1-SWI/SNF complex contained some additional unidentified components.

Experiments were performed to determine whether the additional unidentified components of the BRCA1-SWI/SNF complex represented polypeptides previously shown to be associated with BRCA1. For example, BRCA1 has been reported to be a component of the RNAPII holoenzyme complex (Scully, R. et al. 1997. Proc. Natl. Acad. Sci. USA 94:5605-5610). Using a reconstituted transcription system, the RNAPII transcription activity of the BRCA1-SWI/SNF complex was determined. Results showed that the BRCA1-SWI/SNF complex displayed no RNAPII activity. In fact, Western blot analysis revealed that BRCA1 and RNAPII have different elution profiles. These data demonstrated that the BRCA1-SWI/SNF complex was not a component of a human RNAPII complex.

BRCA1 has also been shown to be in a stable complex with human Rad50-hMrell-p95 complex (Zhong, Q. et al. 1999. Science 285:747-750). Although some components of this complex co-purified with BRCA1 in earlier steps of the purification, Western blot analysis of the Superose 6 column fractions indicated that Rad50/Mre11/095 are components of a larger complex.

In addition, the BRCA1-SWI/SNF was devoid of the BRCA2 protein. Considered together, these data demonstrated that the BRCA1-SWI/SNF complex of the present invention is a different complex that of previously described BRCA1-containing complexes.

To further examine the association of the SWI/SNF complex with the BRCA1 protein, SWI/SNF complexes were purified from Ini-11 cells as previously described (Phelan, et al. 1999. Mol. Cell 3:247-253). This HeLa-derived cell line expresses Flag-SNF5, a Flag-tagged component of SWI/SNF complex. SNF5 has been reported to be a core component of a number of SWI/SNF complexes in mammalian cells (Wang, W. et al. 1996. Embo J. 15:5370-5382; Wang, W. et al. 1996. Genes Dev. 10:2117-2130). Nuclear extract from Ini-11 cells or untagged-HeLa cells was affinity purified using anti-Flag antibodies followed by elution of the bound material with Flag peptide. Western blot analysis of the affinity eluates revealed the specific association of BRCA1 with the tagged-SWI/SNF complexes. Further fractionation of the Flag-affinity eluate by Superose 6 gel filtration and Western blot analysis of column fractions demonstrated the association of BRCA1 with a 2 MDa SWI/SNF complex.

Having determined that BRCA1 is intimately associated with SWI/SNF complexes, experiments were performed to determine which subunit of the complex (i.e., Flag-BRG1 or the trimeric Flag-SNF5/BAF155/BAF170 complex) mediated the interaction with BRCA1. Flag-BRG1 or the trimeric FlagSNF5/BAF155/BAF170 complex was produced in insect cells and purified to homogeneity. Full-length his-tagged BRCA1 was also produced in insect cells and purified through a nickel column. The Flag-BRG1 or the trimeric Flag-SNF5/BAF155/BAF170 was then incubated with his-BRCA1 and the protein mix was then purified through a Flag affinity column. Bound proteins were eluted using Flag peptide and analyzed by Western blotting using anti-BRCA1, anti-BRG1 and anti-SNF5 antibodies. Results showed that only BRG1 interacted directly with BRCA1. Addition of the trimeric complex to the BRG1/BRCA1 reaction resulted in a slight increase in the recovery of BRCA1 and BRG1. These data demonstrated that there is a direct interaction between BRCA1 and the BRG1 protein. Similar results were obtained when his-BRCA1 and Flag-BRG1 were coexpressed in insect cells and the BRG1/BRCA1 complex was purified using an anti-Flag affinity column.

To determine which domain of BRCA1 interacts with BRG1, protein-protein interaction studies were performed using GST-BRCA1 constructs spanning the open-reading frame as have been previously described (Scully, R. et al. 1997. Cell 88:265275). This analysis revealed a specific interaction of BRG1 with a fragment of BRCA1 spanning the amino acids 260-553 encoded within exon 11.

With the interaction of BRCA1 and the BRG1 subunit of the SWI/SNF complex defined, studies were performed to examine the functional consequence of this BRCAL association, i.e., control of transcription activation. BRG1 is known to contain a DNA-dependent ATPase activity that is an essential component of the SWI/SNF chromatin remodeling activity (Khavari, P. A. et al. 1993. Nature 366:170-174). Therefore, it was believed that overexpression of the dominant negative ATPase mutant of BRG1 would functionally interfere with BRCA1-mediated transcription activity. Transient transfection of BRCA1 resulted in a 3-fold stimulation of p53-dependent transcription. This action was specific as neither the mutant BRCA1 lacking exon 11 or transfection of BRCA1 in absence of p53 resulted in stimulation of transcription activity. The effect of increasing concentrations of mutant or wild-type BRG1 on BRCA1-dependent transcription was examined to determine whether BRCA1-dependent stimulation of transcription was mediated through the SWI/SNF complex. While addition of varying concentrations (0.5 to 4 micrograms) of mutant BRG1 completely abolished BRCA1-mediated transcription, only higher concentrations of wild-type BRG1 (2 to 4 micrograms) displayed an inhibitory effect. Co-transfection of the mutant BRG1 (5 microgram) did not affect the TAT (HIV transactivator) mediated stimulation of transcription and only slightly reduced the TAX (HTLV-I transactivator)-mediated transcription. Considered together, these results demonstrated a functional association of BRCA1 and the SWI/SNF-related complex.

The functional analysis was extended by examining the role of BRCA1-SWI/SNF complex in activation of endogenous p53-responsive promoters. There was a pronounced p53-dependent stimulation of the p21 and p53 promoters by exogenously expressed BRCA1. The p53-dependent enhancement of transcription by BRCA1 was completely and specifically abolished by addition of the dominant negative mutant of BRG1. These results demonstrate a functional association of BRCA1 and the SWI/SNF complex.

For the first time, the BRCA1 protein has been shown to form a stable complex with a SWI-SNF-related chromatin remodeling complex. This complex was shown to be devoid of RNAPII, any components of the Rad50 complex, and BRCA2, defining the BRCA1-SWI/SNF complex of the present invention as separate from any form of the RNAPII holoenzyme or the Rad50/Mre11/p95 complex. Further, the interaction of BRCA1 has been shown to be mediated by the BRG1 protein subunit of SWI/SNF. Studies have also shown that the previously reported p53-mediated co-activation function of BRCA1 is mediated through the SWI/SNF complex. Additionally, the BRCA1-SWI/SNF complex of the present invention has been resolved to near homogeneity as a complex of 18 polypeptides that include BRG1, BAF170, BAF155, BAF60, and SNF5.

Using the information on the identity of the polypeptides that make up the BRCA1-SWI/SNF complex, experiments can be performed to isolate their genes. It is possible that the sequences of the polypeptides will correspond to human genes that have already been sequenced. Therefore, databases will be screened to identify such genes using methods known to those of skill in the art. Further, once the polypeptides have been identified and sequenced, they can be expressed in cells. For example, using methods known to those of skill in the art, the BRCA1-associated polypeptides can be overexpressed in E. coli as well as insect cells. With production of a recombinant protein, monoclonal antibodies can then be generated. Immunolocalization experiments will then be possible using the antibodies to the novel polypeptides. The chromosomal location of the new gene products is also of interest as they may correspond to hot spots for the development of cancer.

Since data have demonstrated that BRCA1 stimulation of p53 transcriptional activity is mediated through the activity of the SWI/SNF complex, studies will also be performed to examine the function of this complex in estrogen receptor transcriptional responsiveness. The role of the BRCA1-SWI/SNF complex in estrogen responsiveness can be characterized by examining estradiol-stimulated activation of a estrogen-responsive reporter. The contribution of this complex in activation of known endogenous estrogen responsive promoters will then be tested. Chromatin-immunoprecipitations can also be performed to directly examine the binding of BRCA1-SWI/SNF to these estrogen-responsive promoters. Chromatin-immunoprecipitation experiments can also be performed to examine the promoter occupancy of the estrogen responsive genes by the BRCA1-SWI/SNF complex. Since estrogen stimulation of mammary epithelia is thought to be a major factor in promoting development of breast cancer, a role for this complex in estrogen signaling would be an important discovery.

The following non-limiting examples are provided to further illustrate the present invention.

### EXAMPLES

### Example 1

### Chromatographic Purification of the BRCA1 Complex

Following cell breakage, nearly all BRCA1 is recovered in the soluble cell fraction. The BRCA1 complex was purified from 2 grams of HeLa nuclear extract. Nuclear extract was loaded on a 250 ml column of phosphocellulase (P11, Whatman) and fractionated stepwise by 0.1, 0.3, 0.5 and 1.0 M KCl in buffer A (20 mM Tris HCL, pH 7.9, 0.2 mM EDTA, 10 mM betaME, 10% glycerol, 0.2 mM PMSF). The P11 0.5 M KCl fraction (250 mg) was loaded on a 45 ml DEAE-Sephacel column (Pharmacia) and eluted with 0.35 M KCl. The 0.35 M KCl elution (140 mg) was dialyzed to 700 mM NH₄SO₄ in buffer HB (20 mM Hepes, pH 7.6, 4 mM dithiothreitol, 0.5 mM EDTA, 10% glycerol, 0.5 mM PMSF, 1 μg/ml aprotinin, 1 μg/ml leupeptin, and 1 μg/ml pepstatin) and loaded on a Phenyl SUPEROSE HR 10/10. The column was resolved using a linear 10 column volume gradient of 700 to 0 mM NH4S04 in buffer HE. BRCA1-containing fractions were dialyzed to 10 mM KPO₄ in buffer HA (5 mM Hepes, pH 7.6, 1 mM dithiothreitol, 0.5 mM PMSF, 10 μM CaCl₂, 10% glycerol, 40 mM KCl, 1 μg/ml aprotinin, 1 μg/ml leupeptin, and 1 μg/ml pepstatin) and loaded on a BioScale CHT5-I column (Biorad). The column was resolved using a linear 15 column volume gradient of 10 to 600 mM KPO₄ in buffer HA. Fractions containing BRCAL were dialyzed to 100 mM KCl in buffer A containing 1 μg/ml aprotinin, 1 μg/ml leupeptin, and 1 μg/ml pepstatin, and then loaded on Mon Q HR 5/5 (Pharmacia). The column was resolved using a linear 10 column volume gradient of 100 to 500 KCl in buffer A containing 1 μg/ml aprotinin, 1 μg/ml leupeptin, and 1 μg/ml pepstatin. BRCA1-containing fractions were fractionated on a Superose 6 HR 10/30 (Pharmacia) equilibrated in 0.7 M KCl in buffer A containing 0.1% NP-40 and 1 μg/ml aprotinin, 1 μg/ml leupeptin, and 1 μg/ml pepstatin.

### Example 2

### Immunoaffinity Purification of BRCA1 Complex

HeLa nuclear extract (1.2 g) was fractionated according to the protocol described above using P11 and DEAE-Sephacel columns. The DEAE-Sephacel pool was then dialyzed to 150 mM KCl in buffer D (20 mM Hepes, pH 7.9, 0.25 mM EDTA, 20% glycerol and 0.1% Tween 20). C20 antibodies (300 to 500 μg; Santa Cruz) were cross-linked to Protein A-Sepharose (1 ml; Repligen) using standard techniques (Harlow, E. and D. Lane. 1988. Antibodies: A Laboratory Manual, Cold Spring Harbor Laboratory Press: New York) for affinity purification of BRCAL complex. The fractionated nuclear extract (DEAE-SEPHACEL, 10 mg) was incubated with 1 ml of antibody-Protein A beads for 4 to 5 hours in buffer D at 4 C. The beads were washed twice with 1 M KCl in buffer D followed by two washes with 100 mM KCl in buffer D. The proteins were eluted with either 0.1 M glycine, pH 2.5, and neutralized with 1/10 volume 1.0 M Tris-HCL, pH 8.0, or overnight incubation with C20 peptide (4 mg/ml; corresponding to the last 20 amino acids of BRCA1) in buffer D.

### Example 3

### Affinity Purification of Human SWI/SNF Complexes

HeLa S3 cells expressing Flag-tagged SNF5 (Ini-11) were used for the affinity purification of hSWI/SNF as described (Sif, S. et al. 1999. Genes Dev. 12:2842-2851).

### Example 4

### Mass Spectrometric Peptide Sequencing

Excised bands were subjected to gel reduction, carboxyamidomethylation, and tryptic digestion (Promega). Multiple peptide sequences were determined in a single run by microcapillary reverse-phase chromatography (a custom New Objective 50 μm column terminating in a nanospray 15 μm tip) directly coupled to a Finnigan LCQ Deca quadrupole ion trap mass spectrometer. The ion trap was programmed to acquire successive sets of three scan modes consisting of: full scan MS over alternating ranges of 395-800 m/z or 800-1300 m/z, followed by two data dependent scans on the most abundant ion in those full scans. These dependent scans allowed the automatic acquisition of a high resolution (zoom) scan to determine charge state and exact mass, and Ms/MS spectra for peptide sequence information. The MS/MS spectra were acquired with a relative collision energy of 30%, an isolation width of 2.5 Dalton and dynamic exclusion of ions from repeat analysis. Interpretation of the resulting MS/MS spectra of the peptides was facilitated by programs developed in the Harvard Microchemistry Facility (Chitum, H. S>et al. 1998. Biochemistry 37:10866-10870) and by database correlation with the algorithm SEQUEST (Eng, J. K. et al. 1994. J. Am. Soc. Mass Spectrom. 5:976-989).

### Example 5

### Mononucleosome DNase I Cleavage Assay

Assay of STP-dependent disruption of mononucleosomes was performed in accordance with well known methods for example, Owen-Hughes, T. et al. (1996. Science 273:513-516).

### Example 6

### Immunoblot Analysis

Anti-BRG1, BAF170, BAF155, BAF60b, and SNF5 antibodies are available (Wang, W. et al. 1996. Embo J. 15:5370-5382). Immunoblotting with alkaline phosphatase was performed in accordance with well known methods. For example, as described by Bochar, D. A. et al. (2000. Proc. Natl. Acad. Sci. USA 97:1038-1043).

### Example 7

### Protein-Protein Interaction Studies

Recombinant proteins (rBAF155B, rBAF170, rFlag-SNF5, rFlag-BRG1, and rHis-BRCA1) were expressed in a baculovirus expression system. Insect cells (Sf21) were infected with one recombinant virus (rFlag-BRG1, or rHis-BRCA1) or co-infected with multiple viruses (rBAF155B, rBAF170, rFlag-SNF5). The infected cells (10 multiplicity of infection and 48 hours post infection) were harvested and sonicated in 100 mM KCl in buffer A. The cell lysates were cleared by centrifugation (20,000×g for 20 minutes at 4 C). The Fal-tagged proteins (rFlag-BRG1 or rBAF155B/rBAF170/rFlag-SNF5 protein complex) were affinity purified with anti-Flag M2 affinity gel (Sigma) according to the manufacturer's protocol except that buffer A with 100 mM KCl, 300 mM KCl and 100 μg Flag peptide in 100 mM KCl was used in binding, washing and elution steps respectively. Rhis-BRCA1 was purified using Ni-NTA Agaraose (Qiagen) as recommended y the manufacturer except that 500 mM KCl in buffer A without EDTA was used.

Protein-protein interactions were detected using an affinity bound Flag tagged protein or protein complex. A target protein or protein complex in 100 mM KCl in buffer A was added to the anti-Flag M2 affinity gel bound protein or protein complex and allowed to interact with the gel bound protein for 1 hour at 4 C with constant inversion. Non-specifically bound proteins were removed by repeated washing with 300 mM KCl in buffer A. Bound proteins were eluted with 100 μg Flag peptide in 100 mM KCl in buffer A and analyzed by Western blotting using the indicated antibodies.

### Example 8

### GST-BRCA1 Fusion Proteins and GST Pull-Down Assay

GST-BRCA1 fusion proteins constructs were made in accordance with known methods (Scully et al. 1997. Cell 88:265-275). GST-BRCA1 fusion proteins #1 to #6 correspond to amino acids 1-324, 260-553, 502-802, 758-1064, 1005-1313, and 1314-1863, respectively. GST and GST-BRCA1 fusion proteins were expressed in E. coli BL21. Cells were harvested and lyses by sonicating in 150 mM NaCl in buffer G (50 mM Tris, pH 8.0, 10% glycerol, 0.5% Triton X-100, 0.5 mM PMSF, 1 μg/ml aprotinin, 1 μg/ml leupeptin, and 1 μg/ml pepstatin). The cell lysates were cleared by centrifugation (105,000×g for 60 minutes at 4 C). Concentration of GST or GST-BRCA1 fusion proteins in cell lysates was determined by estimation of protein concentration by Coumassie staining of SDS-PAGE samples purified by affinity chromatography on glutathione-Sepharose (Amersham Pharmacia). Cell lysates containing approximately 5 μg of GST-BRCA1 fusion protein were mixed with 10 μl glutathione-Sepharose, incubated for 3 hours at 4 C, and washed 3 times each with 150 mM NaCl, 500 mM NaCl, and 150 mM NaCl in buffer G. Approximately 5 μg of recombinant BRG1 was added and beads were incubated for 3 hours at 4 C. Beads were washed 2 times each with 500 mM NaCl and 150 mM NaCl in buffer G and eluted with 30 mM glutathione plus 150 mM NaCl in buffer G. Samples were subjected to SDS-PAGE followed by Western blotting to determine the presence or absence of BRG1.

### Example 9

### Transient Transfection

Lymphocyte (CEM, 12D7) cells were grown to mid log phase and were prepared for DNA electroporation as described with slight modification (Kashanchi, F. et al. 1992. Nucleic Acids Res. 20:4673-4674). Cells (5×10⁶) were electroporated at 230 volts and plated in 10 ml of complete RPMI-1640 media for 18 hours prior to harvest and CAT assay. Super-coiled double banded cesium chloride plasmids (pCMV-p53, G5p53-CAT) for lymphocyte transfection are available in the art (Muralidhar, S. et al. 1996. J. Virol.70:8691-8700). Titration of the reporter was initially performed to obtain a reproducible CAT enzymatic assay and subsequently 5 μg of the reporter was used for each transfection assay. BRCA1 and BRG1 constructs are known to one of skill (Jansen, D. E. et al. 1998. Oncogene 16:1097-1112; Khavari, P. A. et al. 1993. Nature 366:170-1740).

### Example 10

### RNase Protection

Cells (293) were grown to mid log phase and transfected at 2.5×10⁶ cell/10 cm plate. Five micrograms of each DNA was mixed with double distilled water in a final volume of 450 μl. Fifty microliters of 2.5 M CaCl₂ was added to the mixture. Five hundred microliters of 2× Hepes (pH 7.16) was added drop-wise to the solution. After incubation at room temperature for 15 minutes, the solution was distributed evenly over the cells. After 4 hours at 37 C, media was replaced by complete DMEM. Cells were harvested 24 hours later.

24 hours after transfection, RNA was isolated with RNAzol reagent (TEL-TEST, Inc.). Each plate was treated with 0.5 ml of RNAzol. Samples, 300 microliters, were chloroform extracted and RNA was precipitated with an equal volume of isopropanol. Pellets were washed with 70% ethanol and suspended in DPEC-treated water at a final concentration of 5 micrograms/liter.

RNase protection assays were performed using the hStress-1 multi-probe template of the RiboQuant Multi-Probe RPA kit (PharMingen). Labeled RNA probes were generated using T7 polymerase and [α-³²P]UTP. 15 micrograms of cellular RNA samples, 5 micrograms of HeLa control RNA, or 10 micrograms of yeast tRNA were separately hybridized with labeled RNA probes. The mixture was incubated at 95 C cooled gradually to 56 C overnight. The incubation temperature was further decreased to 37 C over 30 minutes prior to Rnase digestion. Rnase A (19.2 ng) and Rnase T₁ (60 units) were added to each sample and incubated at 30 C for 45 minutes. After termination of Rnase digestion, RNA was preincubated and suspended in 5 microliters of loading buffer. Samples were run in a 6% TBE/Urea gel (Novex, Inc.) At 100 Watts. Dried gels were exposed to a Phosphoro-Imager cassette (Molecular Dynamics) overnight. Ratios of L32 and GAPDH RNA controls from different samples were compared to 293 mock samples. Counts obtained from protected fragments were divided by the ratios (L32+GADPH) to get corrected values.

### Example 11

### Examining the Role of BRCA1 and the BRCA1-SWI/SWF Complex in Estrogen Signaling

Experiments will determine whether SWI/SNF functions as a co-activator for the estrogen receptor. MCF-7 cells will be transfected with ERE-TK-Luc promoter. Different concentrations of the reporter will be titrated to determine its activity levels in the absence and presence of estradiol in order to determine what concentration of reporter plasmid will yield activation. To examine the contribution of SWI/SNF on estrogen responsiveness, different concentrations of BRG1, Brahma or the ATPase mutant of BRG1 (K783R) will be transfected. Estrogen responsiveness will be measured.

The effect of estrogen on SW13 and C33A cells will be examined, as these cells are known to be devoid of BRG1 and human Brahma. The cells will be stimulated with estradiol following transfection with the ERE-TK-Luc reporter plasmid and estrogen receptor alpha. If there is no stimulation of transcription by estradiol, SWI/SNF will have been shown to be involved. Then, the transcription defect will be rescued by increasing concentrations of BRG1 or Brahma. The effect of the BRG1 ATPase mutant will also be examined. These data will indicate whether the SWI/SNF complex is required to estrogen signaling and if chromatin remodeling activity of SWI/SNF plays a role in this effect.

Studies will be performed to determine whether any cancer-causing mutation in BRCA1 abolishes the known repressive effects of BRCA1 on estrogen transcriptional activity. Increasing concentrations of BRCA1 or the common cancer-causing mutants of BRCA1 will be tested on estrogen responsiveness in MCF7 cells.

Experiments will be performed to determine whether sequestration of SWI/SNF by over-expressed BRCA1 is involved in BRCA1 repression of estrogen transcription. The transcriptional activity of BRCA1 mutants which are unable to interact with BRG1 will be analyzed. If the BRCA1-BRG1 interaction is required for repression then mutants will lack repressive ability.

Experiments will performed to determine whether BRCA1 interacts directly with estrogen receptors. The ability of full length His-tagged recombinant BRCA1 produced in insect cells to interact with translated estrogen receptors alpha and beta will be examined. If there is a direct interaction then the domain on the receptor for the interaction will be mapped.

Experiments will be performed to examine whether estrogen-responsiveness of endogenous promoters is mediated through the BRCA1-SWI/SNF complex. MCF7 cells will be transfected by BRG1, Brahma, K783R and the BRG1 mutant which is unable to interact with BRCA1. Cells will be transfected in combination with the plasmid expressing GFP. Cells expressing GFP will be sorted before estrogen stimulation. Next, the effect of different endogenously expressed BRG1 or Brahma on estrogen responsiveness will be determined. Similar experiments will be performed in SW13 and C33A cells. These data will establish whether the SWI/SNF complex is required for estrogen responsiveness of cellular estrogen promoters.

What is claimed is:

1. A composition for modulating cellular proliferation in mammalian cells which targets a BRG1 subunit of the SWI/SNF complex in the BRCA1-SWI/SNF complex.

2. The composition of claim 1 wherein chromatin remodeling is modulated.

3. A method for modulating cellular proliferation in mammalian cells comprising interacting BRCA1 with a composition which targets a BRG1 subunit of the SWI/SNF complex.

4. The method of claim 3 wherein chromatin remodeling is modulated.

5. A method of screening for cancer comprising identifying the presence of mutations in the BRG1 subunit of the SWI/SNF complex in the BRCA1-SWI/SNF complex and other SWI/SNF subunits in a biological sample.

6. A method of modulating transcriptional activity in a mammalian cell comprising interacting BRCA1 with the BRG1 subunit of mammalian SWI/SNF protein complex in the BRCA1-SWI/SNF complex so that transcription, and thereby cellular proliferation, is modulated.

7. The method of claim 6 wherein chromatin remodeling is modulated.

8. A composition for modulating estrogen signaling in mammalian cells which targets a BRG1 subunit of the SWI/SNF complex in the BRCA1-SWI/SNF complex.
